(19) United States
(12) Patent Application Publication
(10) Pub. No.: US 2022/0033855 A1
(43) Pub. Date: Feb. 3, 2022

(54) ARRAYED NUCLEIC ACID-GUIDED NUCLEASE OR NICKASE FUSION EDITING

(71) Applicant: Inscripta, Inc., Boulder, CO (US)

(72) Inventors: Andrew Garst, Boulder, CO (US); Christian Siltanen, Boulder, CO (US)

(21) Appl. No.: 17/384,770

(22) Filed: Jul. 25, 2021

Related U.S. Application Data
(60) Provisional application No. 63/058,542, filed on Jul. 30, 2020.

(51) Int. Cl. C12N 15/90; C12N 15/11; C12N 9/22; C12N 15/63

(52) U.S. Cl. CPC C12N 15/902; C12N 15/111; C12N 2310/20; C12N 15/63; C12N 9/22

(57) ABSTRACT

The present disclosure relates to methods for performing arrayed nucleic acid-guided nuclease nickase fusion editing allowing for rapid genotypic/phenotypic correlation without sequencing.

### RELATED CASES

This application claims priority to U.S. Ser. No. 63/058,542, filed 30 Jul. 2020, entitled “Arrayed Nucleic Acid-Guided Nuclease Editing”, which is incorporated herein in its entirety.

### FIELD OF THE INVENTION

The present disclosure relates to methods for performing arrayed nucleic acid-guided nuclease or nickase fusion editing allowing for rapid genotypic/phenotypic correlation without sequencing.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently various nucleases have been identified that allow manipulation of gene sequence; hence, gene function. The nucleases include nucleic acid-guided nucleases, which enable researchers to generate permanent edits in live cells, Editing efficiencies frequently correlate with the concentration of guide RNAs (gRNAs) in the cell. That is, the higher the expression level of gRNA, the better the editing efficiency. Further, it is desirable to be able to perform many different edits in a population of cells simultaneously and to do so in an automated fashion, minimizing manual or hands-on cell manipulation.

There is thus a need in the art of nucleic acid-guided nuclease editing for improved methods for increasing the efficiency of and decreasing the time needed for combinatorial editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to compositions, methods, modules and instrumentation for efficient nucleic acid nuclease- or nickase fusion-guided editing in a large population of cells. Efficient editing requires many excess copies of editing cassettes or editing vectors in the cell nucleus. In order to perform highly-multiplexed editing in a single reaction, it is necessary to co-localize cells with many clonal copies of each editing cassette. The present methods take advantage of oligonucleotide synthesis on solid supports with partitions, where one or more sequence-defined oligonucleotides (e.g., editing cassettes and supplemental oligonucleotides) are synthesized in each partition. The methods require that the spatial integrity of the editing cassettes and edited cells be maintained during synthesis and amplification of the editing cassettes, and during cell delivery, transformation, editing and growth.

Thus, some embodiments provide a method for editing a population of live cells with a library of editing vectors comprising rationally-designed editing cassettes in situ comprising: designing and synthesizing a library of editing cassettes on a substrate wherein each editing cassette comprises a gRNA and a repair template and wherein each different editing cassette is in a different partition; washing in first single-stranded supplemental oligonucleotides encoding at least one promoter and at least one first primer site and at least one region complementary to the editing cassettes; performing PCR in the partitions to produce amplified editing cassettes; releasing the amplified editing cassettes from the substrate in the partition; adding cells to the partition; adding transformation reagents to each partition; transforming the cells with the amplified editing cassettes to produce transformed cells; allowing editing to take place in the transformed cells to produce edited cells; making a replica of the substrate; and phenotyping the edited cells.

Yet other embodiments provide a method for editing a population of live cells with a library of editing vectors comprising rationally-designed editing cassettes in situ comprising: designing and synthesizing a library of editing cassettes on a substrate wherein each editing cassette comprises a gRNA and a repair template and wherein each different editing cassette is in a different partition; washing in first single-stranded supplemental oligonucleotides encoding at least one promoter and at least one first primer site and at least one region complementary to the editing cassettes; releasing the amplified editing cassettes from the substrate in the partition; performing PCR in the partitions to produce amplified editing cassettes; adding cells to the partition; adding transformation reagents to each partition; transforming the cells with the amplified editing cassettes to produce transformed cells; allowing editing to take place in the transformed cells to produce edited cells; making a replica of the substrate; and phenotyping the edited cells.

In either of these embodiments, the partition may be selected from wells on a substrate and aqueous droplets in an immiscible carrier fluid and in some aspects, the wells or droplets have a volume of 10 pL to 10 μL.

In some aspects of either of these embodiments, the cells are bacteria cells, yeast cells, mammalian cells including stem cells or plant cells.

In some aspects of either of these embodiments, the amplified editing cassettes range in size from 250 to 2000 bp in length.

In some aspects of either of these embodiments, second supplemental oligonucleotides comprising a second primer site and at least one region complementary to the editing cassettes are washed into the partitions with the first supplemental oligonucleotides.

In some aspects of either of these embodiments, the first supplemental oligonucleotides further comprise a barcode.

In some aspects of either of these embodiments, the cells are added by growing the cells in the partitions in proximity to the editing cassettes, and in other aspects, the cells are added by distributing cells into the partitions.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simple diagram of a method disclosed herein. FIG. 1B is a depiction of a prior art method for synthesizing editing cassettes, inserting the editing cassettes into vector backbones, transforming cells and forming a library of edited cells. FIG. 1C is a depiction of one embodiment of editing cassette synthesis on a microarray and subsequent processing in situ. FIG. 1D depicts an exemplary method of PCR amplification of an editing cassette and a supplemental oligonucleotide to add a promoter sequence. FIG. 1E depicts an exemplary method for clonal rolling circle amplification of substrate-bound editing oligonucleotides for increasing local clonal copies of the editing cassettes. FIG. 1F depicts an alternative method for assembling and amplifying full-length editing constructs (with, e.g., promoter and barcode elements) from substrate-bound editing cassettes. FIG. 1G is a depiction of an alternative embodiment of editing cassette synthesis on a microarray and subsequent processing in situ. FIG. 1H is a series of charts showing various components used for arrayed editing and the stokes radius.

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

As used herein, the terms “amplify” or “amplification” and their derivatives, refer to any operation or process whereby at least a portion of a nucleic acid molecule is replicated or copied into at least one additional nucleic acid molecule. The additional nucleic acid molecule may include a sequence that is substantially identical or substantially complementary to at least a portion of the template nucleic acid molecule. The template nucleic acid molecule can be single-stranded or double-stranded, and the additional nucleic acid molecule can be independently single-stranded or double-stranded. Amplification may include linear or exponential replication of a nucleic acid molecule. In certain embodiments, amplification can be achieved using isothermal conditions; in other embodiments, amplification may include thermocycling. In certain embodiments, the amplification is a multiplex amplification and includes the simultaneous amplification of a plurality of target sequences in a single reaction or process. In certain embodiments, “amplification” includes amplification of at least a portion of DNA and RNA based nucleic acids. The amplification reaction(s) can include any of the amplification processes known to those of ordinary skill in the art. In certain embodiments, the amplification reaction(s) includes methods such as polymerase chain reaction (PCR), ligase chain reaction (LCR), or other methods.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components-translated in an appropriate host cell.

The terms “editing cassette”, “CREATE cassette” or “CREATE editing cassette” refer to a nucleic acid molecule comprising a coding sequence for transcription of a guide nucleic acid or gRNA covalently linked to a coding sequence for transcription of a repair template or homology arm. “Full-length editing construct” refers to an editing cassette or CREATE cassette with one or more control sequences or other useful sequences such as promoter elements, enhancer elements, primer sites, barcodes, and/or terminators, where the added elements are located on one or more “supplemental oligos” or “supplemental oligonucleotides” that are coupled to the editing cassettes via, e.g., ligation or amplification.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease or nickase fusion enzyme.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the term “nickase fusion” refers to a nucleic acid-guided nickase-(or nucleic acid-guided nuclease or CRISPR nuclease) that has been engineered to act as a nickase rather than a nuclease (e.g., the nickase portion of the fusion functions as a nickase as opposed to a nuclease that initiates double-stranded DNA breaks), where the nickase is fused to a reverse transcriptase, which is an enzyme used to generate cDNA from an RNA template. For information regarding nickase-RT fusions see, e.g., U.S. Pat. No. 10,689,669 and U.S. Ser. No. 16/740,421.

“Nucleic acid-guided editing components” refers to one, some, or all of a nuclease or nuclease fusion enzyme, a guide nucleic acid and a repair template.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

The term “partition” as used herein refers to a well, droplet or other defined physical location. In the present case, different nucleic acids (oligonucleotides) and cellular nucleic acids are sequestered in a partition. Partitioning can be achieved by tethering oligonucleotides to a solid surface, confining oligonucleotides in a solid-walled or liquid-walled vessel, or by spatially positioning oligonucleotides such that diffusion between neighboring oligonucleotides is limited during the timeframe required for a reaction to occur.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible.

As used herein the term “repair template” refers to nucleic acid that is designed to introduce a DNA sequence modification (insertion, deletion, substitution) into a locus by homologous recombination using nucleic acid-guided nucleases or nickase fusions or a nucleic acid that serves as a template (including a desired edit) to be incorporated into target DNA by reverse transcriptase in a nickase fusion editing system.

As used herein the term “selectable marker” or “survival maker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease or nickase fusion editing system. The target sequence can be a genomic locus or extrachromosomal locus.

The terms “transformation”, “transfection” and “transduction” are used interchangeably herein to refer to the process of introducing exogenous DNA into cells.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In some embodiments, a coding sequence for a nucleic acid-guided nuclease or nickase fusion is provided in a vector, referred to as an “engine vector.” In some embodiments, the editing cassette may be provided in a vector, referred to as an “editing vector.” In some embodiments, the coding sequence for the nucleic acid-guided nuclease or nickase fusion and the editing cassette are provided in the same vector. A “viral vector” as used herein is a recombinantly produced virus or viral particle that comprises an editing cassette to be delivered into a host cell. Examples of viral vectors include retroviral vectors, lentiviral vectors, adenovirus vectors, adeno-associated virus vectors, alphavirus vectors and the like.

### Nuclease- or Nickase Fusion-Directed Genome Editing Generally

The compositions, methods, automated instruments described herein are employed to allow one to perform nucleic acid nuclease- or nickase fusion-directed genome editing to introduce desired edits to a population of live bacterial, yeast, plant and animal cells. A nucleic acid-guided nuclease or nickase fusion complexed with an appropriate synthetic guide nucleic acid in a cell can cut the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. Preferably and typically, the guide nucleic acid comprises RNA and the gRNA is encoded by a DNA sequence on an editing cassette along with the coding sequence for a repair template. Covalently linking the gRNA and repair template allows one to scale up the number of edits that can be made in a population of cells tremendously. Methods and compositions for designing and synthesizing editing cassettes (e.g., CREATE cassettes) are described in U.S. Pat. Nos. 10,240,167; 10,266,849; 9,982,278; 10,351,877; 10,364,442; 10,435,715; and 10,465,207; and U.S. Ser. Nos. 16/550,092, filed 23 Aug. 2019; Ser. No. 16/551,517, filed 26 Aug. 2019; Ser. No. 16/773,618, filed 27 Jan. 2020; and Ser. No. 16/773,712, filed 27 Jan. 2020, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or gRNA/nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, in the case of mammalian cells the target sequence is typically a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA). The proto-spacer mutation (PAM) is a short nucleotide sequence recognized by the gRNA/nuclease or nickase fusion complex. The precise preferred PAM sequence and length requirements for different nucleic acid-guided nucleases or nickase fusions vary; however, PAMs typically are 2-7 base-pair sequences adjacent or in proximity to the target sequence and, depending on the nuclease or nickase fusion, can be 5′ or 3′ to the target sequence.

In most embodiments, genome editing of a cellular target sequence both introduces a desired DNA change to a cellular target sequence, e.g., the genomic DNA of a cell, and removes, mutates, or renders inactive a proto-spacer mutation (PAM) region in the cellular target sequence (e.g., thereby rendering the target site immune to further nuclease binding). Rendering the PAM at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion can be codon optimized for expression in particular cell types, such as bacterial, yeast, plant and animal cells. The choice of the nucleic acid-guided nuclease or nickase fusion to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. Nickase fusion enzymes typically comprise a CRISPR nucleic acid-guided nuclease engineered to cut one DNA strand in the target DNA rather than making a double-stranded cut (e.g., to derive a nickase), and the nickase portion is fused to a reverse transcriptase. For more information on nucleases and nickase fusion editing see U.S. Ser. Nos. 16/740,418; 16/740,420 and 16/740,421, all filed 11 Jan. 2020. Here, a coding sequence for a desired nuclease or nickase fusion is typically on an “engine vector” along with other desired sequences such as a selective marker.

Another component of the nucleic acid-guided nuclease or nickase fusion system is the repair template comprising homology to the cellular target sequence. For the present compositions, methods, modules and instruments the repair template is in the same editing cassette as (e.g., is covalently-linked to) the guide nucleic acid and is under the control of the same promoter as the gRNA (that is, a single promoter driving the transcription of both the editing gRNA and the repair template). The repair template is designed to serve as a template for homologous recombination with a cellular target sequence cleaved or nicked by the nucleic acid-guided nuclease or nickase fusion, respectively, as a part of the gRNA/nuclease or nickase fusion complex. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Ser. No. 16/275,465, filed 14 Feb. 2019. In certain preferred aspects, the repair template can be provided as an oligonucleotide of between 20-300 nucleotides, more preferably between 50-250 nucleotides. The repair template comprises a region that is complementary to a portion of the cellular target sequence (e.g., a homology arm(s)). When optimally aligned, the repair template overlaps with (is complementary to) the cellular target sequence by, e.g., about as few as 4 (in the case of nickase fusions) and as many as 20, 25, 30, 35, 40, 50, 60, 70, 80, 90 or more nucleotides (in the case of nucleases). The repair template comprises two homology arms (regions complementary to the cellular target sequence) flanking the mutation or difference between the repair template and the cellular target sequence. The repair template comprises at least one mutation or alteration compared to the cellular target sequence, such as an insertion, deletion, modification, or any combination thereof compared to the cellular target sequence.

As described in relation to the gRNA, the repair template is provided as part of a rationally-designed editing cassette along with a promoter to drive transcription of both the gRNA and repair template. As described below, the editing cassette may be provided as a linear editing cassette (e.g., a full-length editing construct), or the editing cassette may be inserted into an editing vector. Moreover, there may be more than one, e.g., two, three, four, or more editing gRNA/repair template pair rationally-designed editing cassettes linked to one another in a linear “compound cassette” or inserted into an editing vector; alternatively, a single rationally-designed editing cassette may comprise two to several editing gRNA/repair template pairs, where each editing gRNA is under the control of separate different promoters, separate promoters, or where all gRNAs/repair template pairs are under the control of a single promoter. In some embodiments the promoter driving transcription of the editing gRNA and the repair template (or driving more than one editing gRNA/repair template pair) is an inducible promoter. In many if not most embodiments of the compositions, methods, modules and instruments described herein, the editing cassettes make up a collection or library editing gRNAs and of repair template pairs representing, e.g., gene-wide or genome-wide libraries of editing gRNAs and repair templates.

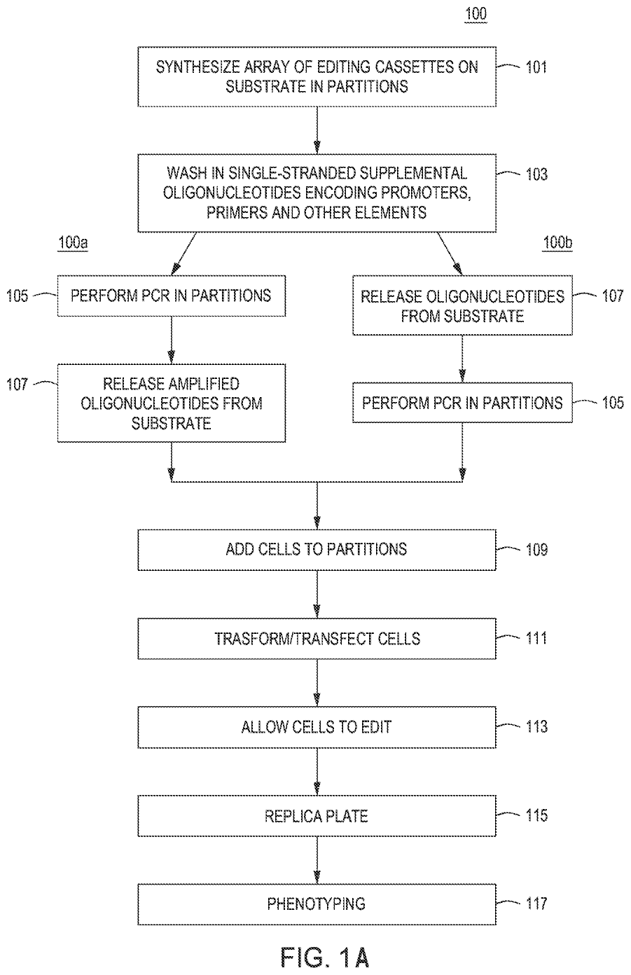

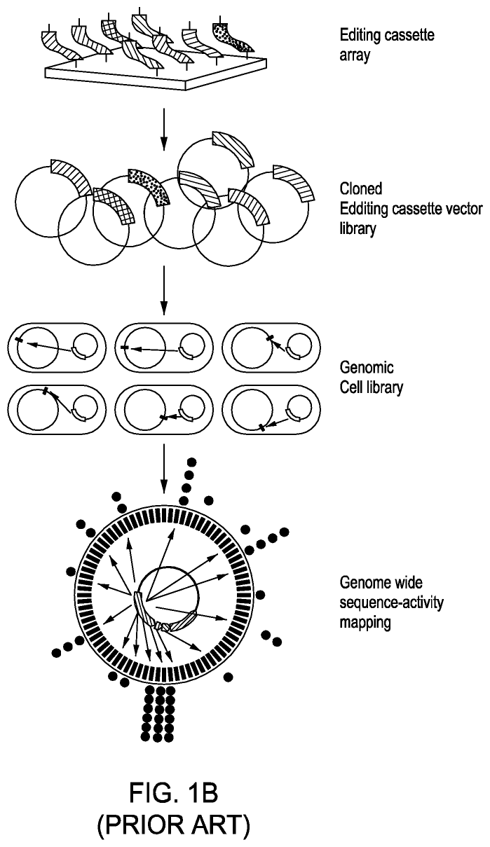

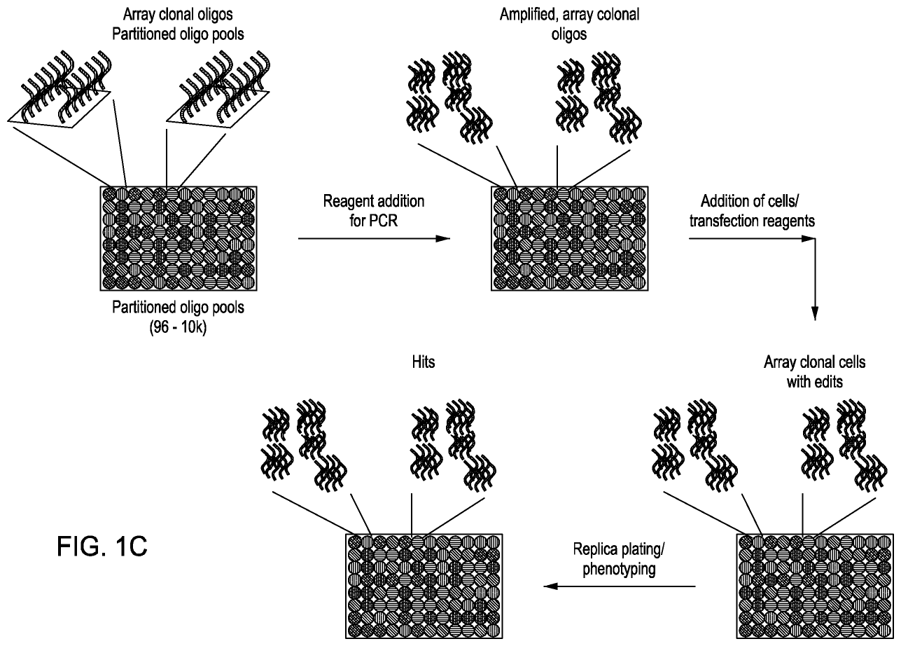

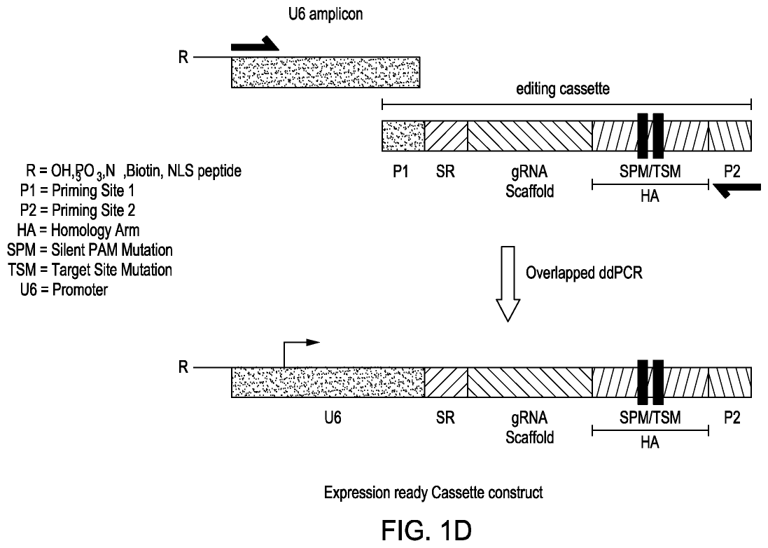

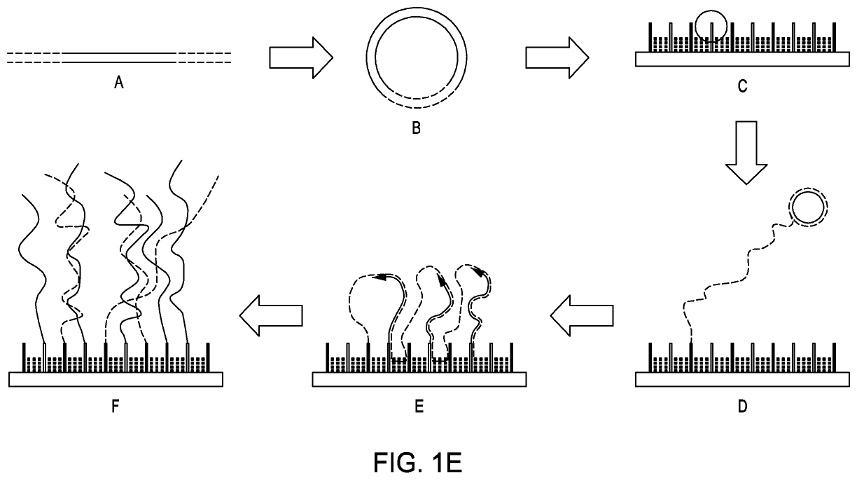

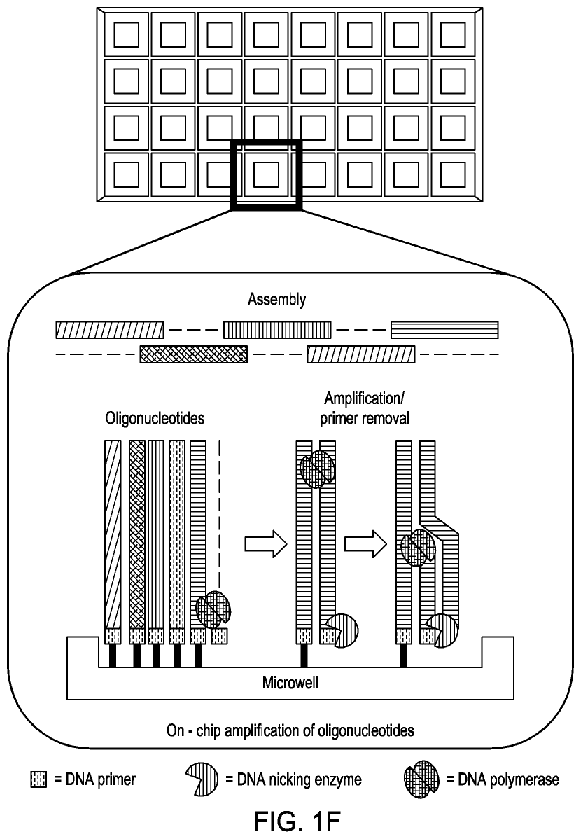

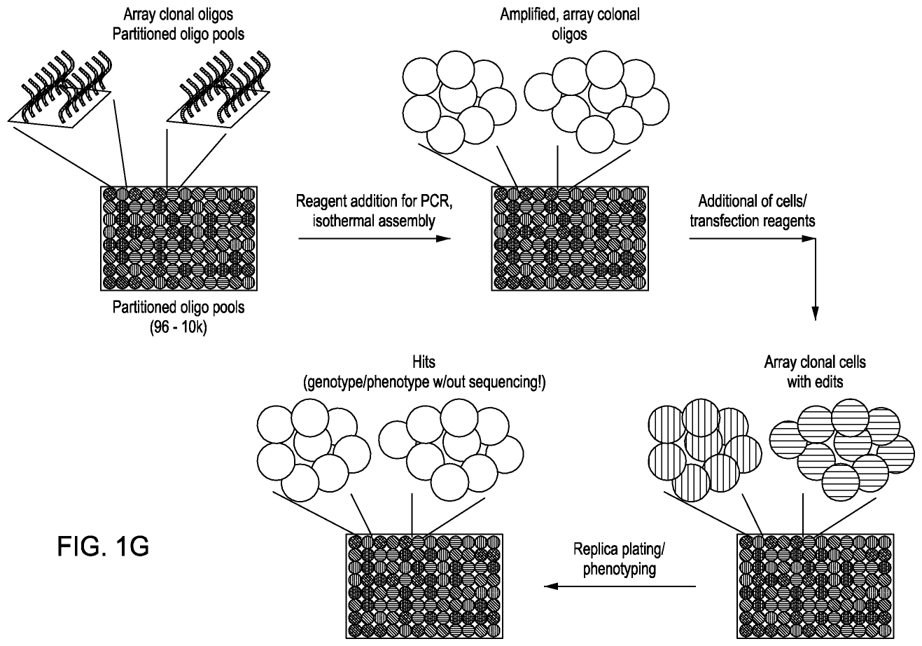

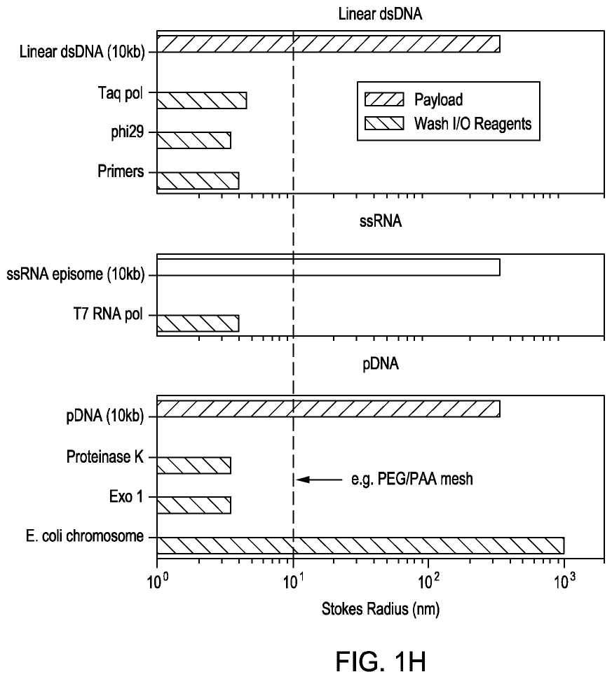

In addition to the repair template, the editing cassettes comprise one or more primer binding sites to allow for PCR amplification of the editing cassettes. The primer binding sites are used to amplify the editing cassette by using oligonucleotide primers as described infra (see, e.g., FIG. 1B), and may be biotinylated or otherwise labeled. In addition, the editing cassette may comprise a barcode. A barcode is a unique DNA sequence that corresponds to the repair template sequence such that the barcode serves as a proxy to identify the edit made to the corresponding cellular target sequence. The barcode typically comprises four or more nucleotides. Also, in preferred embodiments, an editing cassette or editing vector or engine vector further comprises one or more nuclear localization sequences (NLSs), such as about or more than about 1, 2, 3, 4, 5, 6, 7, 8, 9, 10, or more NLSs.

### Editing Cassette Synthesis, Amplification, Cell Transformation and Editing

FIG. 1A is a simple process diagram for a method 100 for nucleic acid-guided nuclease or nickase fusion-guided editing in live cells. In the present methods, the cells of interest are often grown in culture for several passages before the editing cassette synthesizing and amplifying processes shown in FIG. 1A and described herein begin. Cell culture is the process by which cells are grown under controlled conditions, almost always outside the cell's natural environment.

Microbial cell culture—e.g., culturing bacteria and yeast-typically involves isolating a single cell, then propagating that single cell (or clonal cell population) in a defined growth medium that supplies essential nutrients such as amino acids, carbohydrates and certain additives depending on the cell propagated. The type of growth medium will vary depending on whether the cells are prokaryotic (e.g., bacteria) or eukaryotic (yeast) and from genus to genus within prokaryotes and eukaryotes. Cell culture includes growth in a liquid culture, in which cells are suspended and grown in a liquid medium such as Luria Broth, often with shaking/aeration. Liquid cultures are used to grow large amounts of cells. Cell culture also includes growth on agar-based growth medium and, depending on the cells, the growth medium also contains various additives such as antibiotics for cells comprising an antibiotic resistance gene. Culture in either liquid medium or on solid medium typically takes place at 37° C.; however, some thermophilic bacteria from genera, e.g., Bacillus and Thermus are grown at temperatures from 50° C. to 70° C. and other thermophilic bacteria from genera, e.g., Thermococcus and Pyrococcus are grown at temperatures from 70° C. to 100° C. Bacteria of interest include bacteria of the genus Thiomicrospira, Succinivibrio, Candidatus, Porphyromonas, Acidaminococcus, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, Oleiphilus, Omnitrophica, Parcubacteria, and Campylobacter. Yeast of interest include yeast of the genus Ambrosiozyma, Cryptococcus, Candida, Brettanomyces, Pachysolen, Arthroascus, Pachytichospora, Citeromyces, Pichia, Clavispora, Saccharomyces, Cyniclomyces, Saccharomycopsis, Debaryomyces, Schwanniomyces, Dekkera, Sporopachydermia, Guilliermondella, Stephanoascus, Hansenula, Torulaspora, Issatchenkia, Wickerhamiella, Kluyveromyces, Lodderomyces, Wingea, and Zygosaccharomyces.

Plant cells may be used in the methods described herein. Plant cells typically are cultured in simple vessels such as petri dishes; however, such cultures require maintenance in growth rooms that control parameters such as temperature and lighting. See, e.g., McConnick et al., Plant Cell Reports 5:81-84 (1986) for methods and materials related to plant cell culture. Plants of interest include gymnosperms, angiosperms, monocots and dicots, and genera of interest include Oryza (rice), Maize (corn), Triticum (wheat), Secale (rye), Solanum (tomato, potato), Nicotiana (tobacco), Poa (grasses), Fortunella (citrus), Poncirus (citrus), Eremocitrus (citrus), Microcitrus (citrus), Mentha (mint), Glycine (soybean) and Sorghum.

For mammalian cells, like microbial cells, culture conditions vary for each cell type but generally include a medium and additives that supply essential nutrients such as amino acids, carbohydrates, vitamins, minerals, growth factors, hormones, and gases such as, e.g., O₂ and CO₂. In addition to providing nutrients, the medium typically regulates the physio-chemical environment via a pH buffer, and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passages” generally refers to transferring a small number of cells to a fresh substrate with fresh medium, or, in the case of suspension cultures, transferring a small volume of the culture to a larger volume of medium.

Mammalian cells include primary cells, which are cultured directly from a tissue and typically have a limited lifespan in culture; established or immortalized cell lines, which have acquired the ability to proliferate indefinitely either through random mutation or deliberate modification such as by expression of the telomerase gene; and stem cells, of which there are undifferentiated stem cells or partly-differentiated stem cells that can both differentiate into various types of cells and divide indefinitely to produce more of the same stem cells.

Primary cells can be isolated from virtually any tissue. Immortalized cell lines can be created or may be well-known, established cell lines such as human cell lines DU145 (derived from prostate cancer cells); H295R (derived from adrenocortical cancer cells); HeLa (derived from cervical cancer cells); KBM-7 (derived from chronic myelogenous leukemia cells); LNCaP (derived from prostate cancer cells); MCF-7 (derived from breast cancer cells); MDA-MB-468 (derived from breast cancer cells); PC3 (derived from prostate cancer cells); SaOS-2 (derived from bone cancer cells); SH-SY5Y (derived from neuroblastoma cells); T-047D (derived from breast cancer cells); TH-1 (derived from acute myeloid leukemia cells); U87 (derived from glioblastoma cells); and the National Cancer Institute's 60 cancer line panel NCI60; and other immortalized mammalian cell lines such as Vero cells (derived from African green monkey kidney epithelial cells); the mouse line MC3T3; rat lines GH3 (derived from pituitary tumor cells) and PC12 (derived from pheochromocytoma cells); and canine MDCK cells (derived from kidney epithelial cells).

Generally speaking, there are three general types of mammalian stem cells: adult stem cells (ASCs), which are undifferentiated cells found living within specific differentiated tissues, including hematopoietic, mesenchymal, neural, and epithelial stem cells; embryonic stem cells (ESCs), which in humans are isolated from a blastocyst typically 3-5 days following fertilization and which are capable of generating all the specialized tissues that make up the human body; and induced pluripotent stem cells (iPSCs), which are adult stem cells that are created using genetic reprogramming with, e.g., protein transcription factors.

In parallel with preparing the cells of interest for editing, method 100 begins with synthesizing editing cassettes on a substrate in partitions 101. An “editing cassette” refers to a nucleic acid molecule comprising a coding sequence for transcription of a guide nucleic acid or gRNA covalently linked to a coding sequence for transcription of a repair template or homology arm and preferably linked to a barcode that uniquely identifies the editing cassette. A “full-length editing construct” refers to an editing cassette or CREATE cassette with added elements such as one or more of a promoter element, enhancer element, primer site and/or terminator supplied by a supplemental oligonucleotide.

Oligonucleotide synthesis has been known for over 30 years. The vast majority of oligonucleotides are synthesized on automated synthesizers using phosphoramidite methodology. Phosphoramidite methodology is based on the use of DNA phosphoramidite nucleosides that are modified with a 4,4′-dimethoxytrityl (DMTr) protecting group on the 5′-OH, a β-cyanoethyl-protected 3′-phosphite and appropriate conventional protecting groups on the reactive primary amines in the heterocyclic nucleobase. The four classic protected DNA nucleoside phosphoramidites are benzoyl-dA, benzoyl-dC, iso-butyryl-dG and dT (which requires no base protection). Additionally, both acetyl-dC and dimethylformamidine-dG are now also routinely used. The phosphoramidite approach is carried out almost exclusively on automated synthesizers using controlled-pore glass or polystyrene solid supports. (For a review, see Caruthers, Biochem. Soc. Trans., 39:575-80 (2011).) In some synthesis schemes, supports are held in small synthesis ‘columns’ that act as a reaction vessel. The columns are attached to the synthesizer and phosphoramidite and ancillary reagents are passed through the column in cycles thus extending the oligonucleotide chain.

The oligo synthesis cycle consists of four steps: deblocking (detritylation); activation/coupling; capping; and oxidation. Synthesis typically occurs in the 3′ to 5′ direction; which is in fact opposite to enzymatic synthesis by DNA polymerases. Conventionally, the 3′ base in the sequence is incorporated by use of a base-functionalized controlled pore glass (CPG) or polystyrene (e.g., macoporous polystyrene (MPPS)) support. Synthesis initiates with removal (‘deblocking’ or ‘detritylation’) of the 5′-dimethoxytrityl group by treatment with acid (classically 3% trichloroacetic acid in dichloromethane) to make available the reactive 5′-OH group. The phosphoramidite corresponding to the second base in the sequence is activated (using a tetrazole-like product such as 5-(Ethylthio)-1-H-tetrazole or 5-(Benzylthio)-1-H-tetrazole), then coupled to the first nucleoside via the 5′-OH to form a phosphite linkage. Solid phase phosphoramidite coupling usually proceeds to around 99% efficiency: however, if the 1% of molecules remaining with reactive 5′-OH groups are left untreated, unwanted side-products will result. To prevent these side products, a ‘capping’ step is introduced prior to the oxidation to acetylate the unreacted 5′-OH. Capping is accomplished using a solution containing acetic anhydride and the catalyst N-methylimidazole. Unless blocked, these truncated oligos can continue to react in subsequent cycles giving near full-length oligos with internal deletions.

The unstable trivalent phosphite triester linkage is then oxidized via an iodine-phosphorous adduct to a stable pentavalent phosphotriester using iodine in a tetrahydrofuran/(pyridine or lutidine)/water solution. After oxidation, the cycle is repeated, starting with detritylation of the second molecule and so on. The synthesis cycle continues to be repeated until the desired length of oligonucleotide is achieved. At this point there are two choices: either the final 5′-DMTr group can be left in place as a purification ‘handle’ or the final 5′-DMTr group can be removed by a final acid treatment. The oligonucleotide can then be cleaved from the solid support using a suitable deprotection solution, e.g. ammonium hydroxide solution at room temperature. If desired, cleavage and deprotection can be carried out simultaneously. In addition to cleaving the support, the cyanoethyl groups are removed from the sugar-phosphate backbone. Nucleobase protection is also removed at this time. The specific cleavage and deprotection conditions will vary from oligo to oligo depending on the nucleobase protection employed and any modifiers present.

In the methods herein, instead of column synthesis of relatively large quantities of oligonucleotides, the editing cassette oligos are synthesized in parallel on a small scale in the wells or partitions of multi-well plates (currently up to 10,000 wells per plate). CPG solid supports are available in a variety of pore sizes and functionalized nucleoside loadings. Three typical pore sizes are 500 Å, 1000 Å, and 3000 Å. Shorter primer molecules (e.g., approximately 20 bases) can be synthesized on the 500 Å support. Medium-length DNA oligonucleotides (20-80 bases) are best synthesized using the 1000 Å support, and for very long sequences (>80 bases) a 3000 Å support is typically used. Most of the methods described utilize long oligos; however, the method depicted in FIG. 1F may utilize shorter oligos that are assembled to produce long full-length editing constructs.

“Universal supports”-meaning a support where there is no nucleobase or modification already present—are particularly useful for plate-based synthesis as the first base at the 3′-end is determined by the first addition in the synthesis cycle thus eliminating the possibility of an incorrect resin being placed in a well. The synthesis starts with a non-nucleosidic linker being attached to the solid support. Non-nucleoside linkers or nucleoside succinates are covalently attached to the reactive amino groups in aminopropyl CPG, long chain aminoalkyl (LCAA) CPG, or aminomethyl MPPS. A phosphoramidite respective to the 3′-terminal nucleoside residue is coupled to the universal solid support in the first cycle of oligonucleotide chain assembly using the standard protocols described supra. The chain assembly is then continued until completion, after which the solid support-bound oligonucleotide is deprotected. Release of the oligonucleotides occurs by the hydrolytic cleavage of a P—O bond that attaches the 3′-O of the 3′-terminal nucleotide residue to the universal linker. (For additional information on universal supports, see, e.g., Scott, et al., Innovation and Perspectives in Solid-Phase Synthesis, Peptides, Proteins and Nucleic Acids, Biological and Biomedical Applications, p. 115-24 (R. Epton, ed.) Mayflower Press; and for linkers and cleavage strategies see Guillier, et al., Chemical Reviews, 100:2091-2158 (2000).)

In the present methods, 96-well, 384-well and 10,000-well (or more) supports may be used. Currently, each well of a 10,000-well support comprises on the order of several femtomoles (10⁻¹⁵ moles) of DNA, resulting in 10⁵-10⁷ identical sequence-defined molecules per well. It should be apparent to one of ordinary skill in the art given the present disclosure that supports with larger wells or partitions will comprise more identical molecules per well, and that the number of oligonucleotides synthesized per well depends on the particular chemistry and synthesizer.

Following synthesis of the editing cassettes (e.g., oligonucleotides coupled to a solid support comprising a gRNA sequence, a repair template sequence and a barcode), the editing cassettes may not be de-coupled from the solid support and instead, supplemental oligos are added to each well 103. To facilitate the assembly of a full-length editing construct from the shorter editing cassettes, supplemental oligonucleotides are designed to contain sequences that overlap with sequences on the editing cassettes so that they may be assembled together to make oligonucleotides from 250 to 2000 bp in length. (See, FIGS. 1D and 1F infra.) Currently there are dozens of different methods using various types of PCR to assemble long single-stranded oligonucleotides. A summary of many of these methods is reviewed by Xiong, et al., FEMS Microbiol. Rev., 32:522-40 (2008) and Ma et al., Curr. Opin. Chem Biol. 16:260-67 (2012), Generally, the methods use single-stranded synthetic oligonucleotides-here, supplemental oligos—with complementary overlapping sequences to sequences on the editing cassettes to assemble the full-length editing constructs using a thermostable polymerase and PCR, where the only differences between the myriad of PCR-based DNA assembly methods is in how the substituent oligonucleotides are designed to be assembled together and the reaction conditions under which they are assembled.

The supplemental oligos comprise a promoter element, at least one and preferably two primer sites, and sequences complementary to sequences on the editing cassettes. In method 100a, the editing cassettes and supplemental oligos are then amplified 105 to create full-length editing constructs, which positions a promoter 5′ of the gRNA/repair template (e.g., homology arm) to drive transcription of the editing cassette. An exemplary method for this step 105 is described in FIG. 1D and the text related thereto.

After PCR is performed 105, the now full-length editing constructs are released or de-coupled from the substrate 107. Exemplary decoupling chemistries are described supra; however, preferred decoupling strategies for the methods herein prioritize two aspects: first, it is crucial that the spatial integrity of the full-length editing constructs be maintained, and second, the decoupling chemistry must be compatible with cell transformation and cell growth in later steps. An alternative to method 100a is presented in method 100b, where the editing cassettes are released from (i.e., de-coupled from) the substrate 107 before PCR is performed in the partitions 105.

Several different strategies for maintaining spatial segregation of cassettes may be used at different steps of the editing workflow. For example, in one embodiment, cassette synthesis and amplification are performed in an array of physical partitions, where each cassette sequence is isolated within a liquid compartment (10 pL to 10 uL) confined by solid walls (e.g. microarray), an immiscible liquid, or an air-liquid interface. Reaction compartments are then addressed individually by liquid dispensing robotics for subsequent reactions. In another embodiment, cassettes and their amplification products are immobilized onto arrayed spots via terminal or internal chemical modifications that render the oligonucleotide tethered to the surface of the solid support. The immobilized spots may be submerged in a single (fluidically-connected) reaction volume and processed in parallel. In another embodiment, cassettes and their amplification products are confined to spatial locations by a size-dependent semi-permeable material. For example, the cassettes may be encapsulated in a polymer with a characteristic pore size smaller than the size of the oligonucleotide cassette, but larger than the molecules required for its amplification (e.g. PCR reagents like enzymes, primers, nucleobases, etc., see FIG. 1H) thereby entrapping amplicons as they are generated inside the polymer network. Similarly, the cassettes may be partially confined within a microwell that is sealed with a semi-permeable membrane that allows transport of smaller molecules between the microwell and a bulk liquid region or flow channel.

To maintain the spatial integrity of editing cassettes during cell delivery and transformation, cells may be dispensed directly into the isolated liquid compartments described above or, in another embodiment, cells may be grown in close proximity to the tethered or encapsulated cassettes which are then subsequently liberated via an external trigger (e.g. chemical, temperature, or light induced). It is necessary to ensure that the liberated cassettes are delivered specifically to target cells (for example by electroporation or chemical transfection) without mixing between partitions. This is may be achieved by introducing a gasket or immiscible fluid to fully isolate the cassettes and target cells during transformation, or by controlling the diffusion rate of cassettes such that cross-contamination between spots/partitions occurs at a significantly slower rate that transformation (e.g. by appropriately spacing array entities, or by inhibiting diffusion rate by, for example, increasing the viscosity of the medium). See also US Pub. Nos. 2012/0258871; 2013/0096033; 2013/0109595; 2016/0138091; 2016/0145677; 2018/0201980; 2018/0328936 and 202000109443, all of which are incorporated by reference for all purposes.

At step 109, the cells of choice-bacterial, yeast, plant, mammalian or other cells—that have been grown are deposited in the partitions on the substrate. Cells may be added separately to the partitions or, preferably, are added to the substrate in a bulk liquid such that at least one and up to 10,000 cells are added to each partition. Again, any manner of cell delivery to the partitions is acceptable as long as the spatial integrity of the full-length editing constructs is maintained.

Fluid transfer to the partitions in the solid substrate may be accomplished by a robotic handling system including a gantry. In some examples, the robotic handling system may include an automated liquid handling system such as those manufactured by Tecan Group Ltd. of Mannedorf, Switzerland, Hamilton Company of Reno, Nev. (see, e.g., WO2018015544A1 to Ott, entitled “Pipetting device, fluid processing system and method for operating a fluid processing system”), or Beckman Coulter, Inc. of Fort Collins, Colo. (see, e.g., US20160018427A1 to Striebl et al., entitled “Methods and systems for tube inspection and liquid level detection”).

Following adding cells to each partition 109, the cells are transformed or transfected 111. Transformation as used herein is intended to include to a variety of art-recognized techniques for introducing an exogenous nucleic acid sequence (e.g., DNA) into a target cell and the term “transformation” as used herein includes all transformation and transfection techniques. Such methods include, but are not limited to, electroporation, lipofection, optoporation, injection, microprecipitation, microinjection, liposomes, particle bombardment, sonoporation, laser-induced poration bead transfection, calcium phosphate or calcium chloride co-precipitation, or DEAE-dextran-mediated transfection. Cells can also be prepared for vector uptake using, e.g., a sucrose or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014).

Several methods are known in the art for transferring DNA into a variety of plant species, such as those described in Glick and Thompson, eds., Methods in Plant Molecular Biology, CRC Press, Boca Raton, Fla. (1993). Representative examples include electroporation-facilitated DNA uptake by protoplasts (see Rhodes et al., Science, 240(4849):204-207 (1988)); treatment of protoplasts with polyethylene glycol (Lyznik, et al., Plant Molecular Biology, 13:151-161 (1989)); and bombardment of cells with DNA-laden microprojectiles which are propelled by explosive force or compressed gas to penetrate the cell wall (see, e.g., Klein, et al., Plant Physiol. 91:440-444 (1989) and Boynton, et al., Science 240(4858):1534-1538 (1988)). Further, plant viruses can be used as vectors to transfer genes to plant cells. Plant transformation strategies and techniques are reviewed in Birch, Ann. Rev. Plant Phys. Plant Mol. Biol., 48:297 (1997) and Forester, et al., Exp. Agriculture, 33:15-33 (1997).

Once transformed, the cells are allowed to edit 113. If any one of the nucleic acid-guided editing components—e.g., the editing cassette, nuclease or nickase fusion coding sequence—is under the control of an inducible promoter, then conditions are provided to induce transcription of the one or more nucleic acid-guided editing components. If the promoters used to drive transcription of the nucleic acid-guided editing components are constitutive, then editing typically commences after cell transformation. The cells are allowed to edit and then to grow to recover from editing, presumably with a genotype and phenotype dictated by the particular edit made to the cells.

Monitoring of cell growth is usually performed by imaging the cells and/or by, e.g., measuring pH of the medium using a medium comprising a pH indicator. For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used. Other phenotyping methods may include impedance spectroscopy, Raman spectroscopy, mass spectroscopy, and cell-based assays including cell-cell interaction studies. Once a sufficient number of cells have grown, replica plates 115 may be made of the original substrate, where again, maintaining the spatial integrity of the editing cassettes and cells is of the upmost importance. Any number of replica plates may be made for, e.g., cell repositories and phenotyping studies. Because the positions of the different editing cassettes are known, in phenotyping studies the intended edit may be correlated directly to phenotype and confirmed, if desired, by sequencing. Additional indexing molecules that correlate to known array positions may also be added to the array at any time to enable pooled phenotyping assays. For example, RNA oligonucleotides, tandem mass tags, or optically encoded barcoding molecules may be added to the partitions in order to correlate intended edits to the edited cells' transcriptomes, proteomes, metabolomes, etc., via pooled analysis.

FIG. 1B is a depiction of a prior art workflow for synthesizing editing cassettes, inserting the editing cassettes into vector backbones, transforming cells and forming a library of edited cells. Editing cassettes are designed in silico and synthesized on a solid support as pools (10⁴-10⁶ individual library members). The editing cassettes from the array-based synthesis are de-coupled from the solid support in a pooled format, PCR amplified and cloned in multiplex to create stable plasmid-based editing vectors comprising a selection gene. The library of editing vectors are used to transform a population of cells, ideally already transformed with a vector coding for the appropriate nucleic acid-guided nuclease or nickase fusion enzyme (and, optionally, a selection gene). Following selection, phenotypic profiling is performed and cells with desired phenotypes are isolated, grown, and the edit producing the desired phenotype is determined by sequencing.

FIG. 1C is a depiction of one embodiment of editing cassette synthesis on a partitioned solid substrate or microarray with amplification cell addition, transformation and editing performed in situ. Synthesis of a library of editing cassettes (e.g., gRNA/HA/barcode/primer sites) on a partitioned substrate allows for spatial control that can be leveraged to maintain genotype-phenotype associations for massively parallel editing and phenotyping workflows. Surface coupled oligonucleotide synthesis is performed in a partitioned format (e.g., 96-10,000 partitions or more) as described supra. Following surface-coupled synthesis of the editing cassettes, the editing cassettes are de-coupled from the substrate in a manner that maintains the spatial integrity of the editing cassettes. Once de-coupled, supplemental oligonucleotides are added to the partitions and PCR is performed to create full-length editing constructs comprising a promoter, gRNA, HA, barcode and primer sites such that each partition comprises many clonal copies of the full-length editing constructs. Note that the steps of de-coupling and addition of the supplemental oligos can be reversed. Cells and transfection agents are then added to the partitions to promote uptake of the clonal full-length editing constructs and the cells are allowed to edit and grow. The substrate cell population can, optionally, be replicated and the cells can be screened for a phenotype of interest. The positional information of the synthesized editing cassettes is used to infer the genotype without the need for sequencing.

FIG. 1D depicts an exemplary method of PCR amplification of an editing cassette and a supplemental oligonucleotide to add a promoter sequence. This scheme allows for the addition of a promoter, in this case the U6 promoter, to the editing cassette to produce an expression-ready full-length editing construct. The editing cassette comprises, from 5′ to 3′, a first priming site (P1), a gRNA spacer region (SR), a gRNA scaffold region, a repair template or homology arm (HA) comprising both a silent PAM mutation (SPM) and a target site mutation (TSM), and a second priming site (P2). A first primer construct comprising the U6 promoter with a region complementary to the first priming site (P1) and a second primer complementary to the second priming site are used to amplify the editing cassette, resulting in a full-length editing construct comprising from 5′ to 3′, the U6 promoter, the first priming site (P1), the gRNA spacer region (SR), the gRNA scaffold region, the repair template or homology arm (HA) comprising both the silent PAM mutation (SPM) and the target site mutation (TSM), and the second priming site (P2). In addition to the U6 promoter, the U6 primer may comprise other functional or non-functional groups (here, denoted by “R”) such as a phosphate group, an amine group, a biotin tag, a barcode and/or an NLS peptide. This method is specifically adapted for applications in mammalian cell lines where linear DNA templates have been demonstrated to support sufficient expression levels of gRNA and nickase to drive efficient gene editing. After amplification, the amplified editing cassettes are optionally inserted into a vector backbone.

FIG. 1E shows an alternative to amplifying linear editing cassettes in situ. Instead, clonal full-length editing construct clusters are generated on the substrate surface via a rolling circle amplification where clonal copies of the full-length editing constructs are generated. Cluster generation by clonal rolling circle amplification starts with the generation of a single-stranded circular DNA library comprising editing cassettes. The protocol includes steps well known in the art from NGS library formation including DNA fragmentation, end repair of DNA fragments, and the ligation of adapters. In addition to the standard NGS library process, the fragments are circularized by ligase reaction followed by DNA denaturation to get single-stranded circular DNA of the single-stranded circular DNA library. Both strands (e.g., the (+) strand and (−) strand) are present in the single-stranded circular DNA library but bind independently to separate sites on the substrate. Two primers are immobilized onto the surface, where one primer (forward primer) is complementary to the adaptor region within single-stranded circular DNA (−) strand and the other primer (reverse primer) is complementary to the adaptor region within single-stranded circular DNA (+) strand.

After hybridization of the single-stranded circular DNA library, DNA is eliminated by washing followed by the addition of a reaction mix comprising polymerase and cluster generation (DNA amplification) is carried out. Because forward and reverse primers are immobilized on the surface, both strands are amplified within a single cluster during the exponential rolling circle amplification reaction on the solid surface. During amplification, the first strand is extended from one of the primers (e.g. forward primer) forming a concatemer complementary to the single-stranded circular target molecule hybridized to the primer. This first strand concatemer folds back and hybridizes to the other primer (e.g. reverse primer) which in turn is elongated to form another concatemer complementary to the first strand product. Reverse strand products hybridize to complementary primers immobilized on the surface so that new forward strand products are synthesized. A DNA cluster is generated on the surface comprising concatemers of (+) and (−) strands of the circle.

In FIG. 1E, linear template molecules with left and right adaptor arms (dotted line) (A) are used for a ligase reaction to form circular template molecules (B). After denaturation of the circular template DNA, the DNA is hybridized to primers immobilized to the solid support (horizontal bar in gray) (C). The (+) DNA strand and (−) DNA strand binds to the primers because, forward (black vertical lines on surface) and reverse primer (red vertical lines on surface) are immobilized to the surface. In addition to a forward and reverse primer, spacer oligonucleotides (dotted vertical lines on surface) are immobilized to the solid support. The spacer oligonucleotides are used to regulate the DNA copy number and the DNA crowding within the cluster. After hybridization, all non-hybridized circles are eliminated by a washing step, and an amplification reaction mixture is added. The substrate is incubated where the first strand is synthesized from the target circle (D) which re-hybridized to the complementary primers immobilized on the solid support. Primer extension then occurs (E). During the reaction, less primers are available for re-hybridization, less single-stranded DNA can re-hybridize and thus the clonal copies remain single-stranded (F).

FIG. 1F is another embodiment for assembling full-length editing constructs from the array- or substrate-bound editing cassettes; here microarray-based oligonucleotide synthesis of both the editing cassettes and supplemental oligonucleotides is used. Like FIG. 1C, FIG. 1F depicts creating full-length constructs from editing cassettes on the support on which the editing cassettes are synthesized. However, in the method depicted in FIG. 1F, instead of many copies of a single-sequence oligonucleotide being synthesized in a partition, two or more different oligonucleotides are synthesized in each partition, including editing cassettes and one to several supplemental oligos. The massive multiplexing capabilities of array-based oligonucleotide synthesis means that tens of thousands of unique oligonucleotide sequences can be synthesized simultaneously on the array surface. Within the partitions, the oligonucleotides necessary to assemble a unique long construct are synthesized, amplified, and assembled within the individual partitions. Using this method effectively reduces the sequence complexity of a localized oligonucleotide pool, which in turn increases the robustness of assembly while also allowing for synthesis multiplexing that can occur in each of the many partitions on the chip. (See, e.g., Quan, et al., Nat. Biotech., 29:449-52 (2011) and Hughes and Ellington, Cold Spring Harb. Prospect. Biol., 2017; 9:a023812.)

FIG. 1F shows gene synthesis from microarray-synthesized oligonucleotides. Constructs are assembled using on-chip synthesis and assembly by including a single priming site into the 3′-end of every oligonucleotide synthesized on the microarray. The oligonucleotides can then be amplified within microwells on the array by incubating with a common primer and a DNA polymerase. The primer sequence is removed from the assembly oligonucleotides using an endonuclease, freeing the oligonucleotides to be assembled together via polymerase chain assembly within the same well.

FIG. 1G is a depiction of an embodiment of editing cassette synthesis on a partitioned solid substrate with amplification and editing performed in situ similar to that shown in FIG. 1C except in this embodiment-instead of creating full-length editing linear—an editing vector is created. As in FIG. 1C, synthesis of a library of editing cassettes (e.g., gRNA/HA/barcode/primer sites) on a partitioned substrate allows for spatial control that can be leveraged to maintain genotype-phenotype associations for massively parallel editing and phenotyping workflows. Surface coupled oligonucleotide synthesis is performed in a partitioned format (e.g., 96-10,000 partitions or more) as described supra. Following surface-coupled synthesis of the editing cassettes, the editing cassettes are de-coupled from the substrate in a manner that maintains the spatial integrity of the editing cassettes. Once de-coupled, the editing cassettes may be amplified, then vector backbones are added to the partitions and isothermal assembly of the editing vectors is performed to create editing vectors comprising a promoter, gRNA, HA, barcode, primer sites, selection genes and other control sequences such that each partition comprises many clonal copies of the editing vectors. As with the method depicted in FIG. 1C, note that the steps of de-coupling and addition of the supplemental oligos can be reversed. Cells and transfection agents are then added to the partitions to promote uptake of the editing vectors and the cells are allowed to edit and grow. The substrate cell population can, optionally, be replicated and the cells can be screened for a phenotype of interest. The positional information of the synthesized editing cassettes is used to infer the genotype without the need for sequencing.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for editing a population of live cells with a library of editing vectors comprising rationally-designed editing cassettes in situ comprising:
designing and synthesizing a library of editing cassettes on a substrate wherein each editing cassette comprises a gRNA and a repair template and wherein each different editing cassette is in a different partition;
washing in first single-stranded supplemental oligonucleotides encoding at least one promoter and at least one first primer site and at least one region complementary to the editing cassettes;
performing PCR in the partitions to produce amplified editing cassettes;
releasing the amplified editing cassettes from the substrate in the partition;
adding cells to the partition;
adding transformation reagents to each partition;
transforming the cells with the amplified editing cassettes to produce transformed cells;
allowing editing to take place in the transformed cells to produce edited cells;
making a replica of the substrate; and
phenotyping the edited cells.

2. The method of claim 1, wherein the partition is selected from wells on a substrate and aqueous droplets in an immiscible carrier fluid.

3. The method of claim 2, wherein the partitions comprise wells on a substrate.

4. The method of claim 3, wherein the wells have a volume of 10 pL to 10 μL.

5. The method of claim 2, wherein the partitions comprise aqueous droplets in an immiscible carrier fluid.

6. The method of claim 1, wherein the cells are bacteria cells.

7. The method of claim 1, wherein the cells are yeast cells.

8. The method of claim 1, wherein the cells are mammalian cells.

9. The method of claim 8, wherein the cells are stem cells.

10. The method of claim 1, wherein the cells are plant cells.

11. The method of claim 1, wherein the amplified editing cassettes range in size from 250 to 2000 bp in length.

12. The method of claim 1, second supplemental oligonucleotides comprising a second primer site and at least one region complementary to the editing cassettes are washed into the partitions with the first supplemental oligonucleotides.

13. The method of claim 1, wherein the first supplemental oligonucleotides further comprise a barcode.

14. The method of claim 1, wherein the cells are added by growing the cells in the partitions in proximity to the editing cassettes.

15. The method of claim 1, wherein the cells are added by distributing cells into the partitions.

16. A method for editing a population of live cells with a library of editing vectors comprising rationally-designed editing cassettes in situ comprising:
designing and synthesizing a library of editing cassettes on a substrate wherein each editing cassette comprises a gRNA and a repair template and wherein each different editing cassette is in a different partition;
washing in first single-stranded supplemental oligonucleotides encoding at least one promoter and at least one first primer site and at least one region complementary to the editing cassettes;
releasing the amplified editing cassettes from the substrate in the partition;
performing PCR in the partitions to produce amplified editing cassettes;
adding cells to the partition;
adding transformation reagents to each partition;
transforming the cells with the amplified editing cassettes to produce transformed cells;
allowing editing to take place in the transformed cells to produce edited cells;
making a replica of the substrate; and
phenotyping the edited cells.

17. The method of claim 16, wherein the partition is selected from wells on a substrate and aqueous droplets in an immiscible carrier fluid.

18. The method of claim 17, wherein the partitions comprise wells on a substrate.

19. The method of claim 18, wherein the wells have a volume of 10 pL to 10 μL.

20. The method of claim 17, wherein the partitions comprise aqueous droplets in an immiscible carrier fluid.

21. The method of claim 16, wherein the cells are bacteria cells.

22. The method of claim 16, wherein the cells are yeast cells.

23. The method of claim 16, wherein the cells are mammalian cells.

24. The method of claim 23, wherein the cells are stem cells.

25. The method of claim 16, wherein the cells are plant cells.

26. The method of claim 16, wherein the amplified editing cassettes range in size from 250 to 2000 bp in length.

27. The method of claim 16, second supplemental oligonucleotides comprising a second primer site and at least one region complementary to the editing cassettes are washed into the partitions with the first supplemental oligonucleotides.

28. The method of claim 16, wherein the first supplemental oligonucleotides further comprise a barcode.

29. The method of claim 16, wherein the cells are added by growing the cells in the partitions in proximity to the editing cassettes.

30. The method of claim 16, wherein the cells are added by distributing cells into the partitions.
